(19) United States
(12) Patent Application Publication
(10) Pub. No.: US 2016/0377533 A1
(43) Pub. Date: Dec. 29, 2016

(54) GAS MEASURING APPARATUS, GAS MEASURING METHOD AND GAS CELL

(71) Applicant: KABUSHIKI KAISHA TOSHIBA, Minato-ku (JP)

(72) Inventors: Miyuki KUSABA, Meguro (JP); Akira Maekawa, Kamakura (JP); Shigeyuki Takagi, Fujisawa (JP); Hiroshi Hasegawa, Yokosuka (JP); Takashi Magara, Meguro (JP); Tsutomu Kakuno, Fujisawa (JP); Yasutomo Shiomi, Koza (JP)

(73) Assignee: KABUSHIKI KAISHA TOSHIBA, Minato-ku (JP)

(21) Appl. No.: 15/259,778

(22) Filed: Sep. 8, 2016

Related U.S. Application Data
(63) Continuation of application No. PCT/JP2015/076992, filed on Sep. 24, 2015.

(30) Foreign Application Priority Data
Jan. 20, 2015 (JP) 2015-008899

(51) Int. Cl. G01N 21/3504; G01N 33/497; G01N 21/05; G01N 21/39

(52) U.S. Cl. CPC G01N 21/3504; G01N 21/39; G01N 33/497; G01N 21/05; G01N 33/004

(57) ABSTRACT

A gas measuring apparatus of an embodiment includes a light source, a gas cell and a detection portion. The light source emits infrared light. Into the gas cell, gas containing ¹³CO₂ and ¹²CO₂ is introduced, and the gas cell includes an incident surface on which the infrared light is incident and an emission surface through which the infrared light is transmitted. The gas cell has a cell length of 2.5 cm or more and 20 cm or less. The detection portion measures a first transmittance of transmitted light from the emission surface at a first wavelength in wavelength range in an absorption line of the ¹³CO₂ and a second transmittance of transmitted light from the emission surface at a second wavelength in the wavelength range in an absorption line of the ¹²CO₂, and is capable of calculating a concentration of each of the ¹³CO₂ and the ¹²CO₂.

### CROSS-REFERENCE TO RELATED APPLICATIONS

This is a continuation application of International Application PCT/JP2015/076992, filed on Sep. 24, 2015. This application also claims priority to Japanese Application No. 2015-008899, filed Jan. 20, 2015. The entire contents of each are incorporated herein by reference.

### FIELD

Embodiments described herein relate generally a gas measuring apparatus, gas measuring method and gas cell.

### BACKGROUND

When using infrared light, the concentrations of a variety of gases contained in breath or an environmental gas can be measured.

In the case of measurement of breath, for example, by measuring the concentrations of gases such as CO₂, CO, NH₃, NO₂, C₂H₂, and CH₄, the presence or absence of abnormality can be known.

However, when the concentrations of gases are high, absorption is saturated, and therefore, the absorption spectra of these gases need to be corrected.

### BRIEF DESCRIPTION OF THE DRAWINGS

FIG. 1A is a schematic front view of a gas measuring apparatus according to the embodiment, and FIG. 1B is a schematic plan view of a gas cell;

FIG. 2A is a graph showing an absorption ratio at a wavenumber of 2200 to 2400 cm⁻¹, and FIG. 2B is a graph showing an absorption ratio at a wavenumber of 2295 to 2297 cm⁻¹;

FIG. 3A is a graph showing the absorption coefficients of ¹³CO₂ and ¹²CO₂ at a wavenumber of 2275 to 2325 cm⁻¹, and FIG. 3B is a graph showing the absorption coefficients at a wavenumber of 2295.7 to 2296.3 cm⁻¹;

FIG. 4 is a graph showing dependence of the relative error of a gas concentration on a transmittance;

FIG. 5 is a flow diagram of the gas measuring method according to the embodiment; and

FIGS. 6A to 6C are schematic views of the QCL.

### DETAILED DESCRIPTION

In general, a gas measuring apparatus of an embodiment includes a light source, a gas cell, and a detection portion. The light source emits infrared light. Into the gas cell, gas containing ¹³CO₂ and ¹²CO₂ is introduced, and the gas cell includes an incident surface on which the infrared light is incident and an emission surface through which the infrared light is transmitted. The gas cell has a cell length of 2.5 cm or more and 20 cm or less. The detection portion measures a first transmittance of transmitted light from the emission surface at a first wavelength in wavelength range in an absorption line of the ¹³CO₂ and a second transmittance of transmitted light from the emission surface at a second wavelength in the wavelength range in an absorption line of the ¹²CO₂, and is capable of calculating a concentration of each of the ¹³CO₂ and the ¹²CO₂.

Embodiments of the invention will now be described with reference to the drawings.

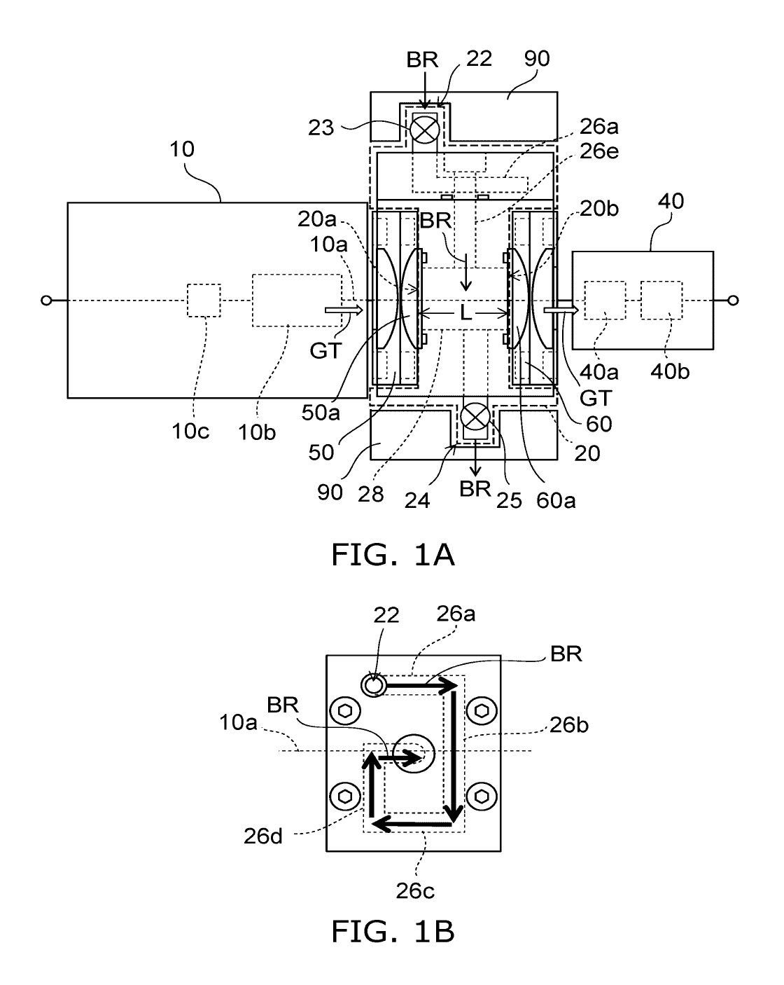

FIG. 1A is a schematic front view of a gas measuring apparatus according to the embodiment, and FIG. 1B is a schematic plan view of a gas cell.

The gas measuring apparatus includes a light source 10, a gas cell 20, and a detection portion 40. The light source 10 emits infrared light which is wavelength tunable in a wavelength range of 4.34 μm or more to 4.39 μm or less. Into the gas cell 20, breath BR or a reference gas is introduced. The breath BR contains ¹³CO₂ and ¹²CO₂, each of which has at least one absorption line in the wavelength range. The gas is not limited to the breath.

The gas cell 20 includes an incident surface 20a and an emission surface 20b with respect to infrared light. An optical axis 10a of incident light I0 of infrared light is orthogonal to each of the incident surface 20a and the emission surface 20b. A cell length L is defined as a distance between the incident surface 20a and the emission surface 20b along the optical axis 10a, and is set to, for example, 2.5 cm or more and 20 cm or less.

The detection portion 40 measures a first transmittance of transmitted light from the emission surface 20b at a first wavelength in the wavelength range in an absorption line of ¹³CO₂ and a second transmittance of transmitted light from the emission surface 20b at a second wavelength in the wavelength range in an absorption line of ¹²CO₂, and is capable of calculating a concentration of each of ¹³CO₂ and ¹²CO₂.

As the light source 10, a QCL (quantum cascade laser), a semiconductor laser, or the like can be used. One of the gases set as a target for the gas measuring apparatus is CO₂. It is possible to make gastric diagnosis based on a change in a ratio of isotopes of CO₂ between before and after a reagent is taken. The infrared light from a QCL or a semiconductor laser is a laser and is preferred because it is easily converted to parallel light and is absorbed by CO₂ gas with high efficiency.

A concentration of CO₂ contained in human breath is from about 0.5 to 8%. Further, CO₂ has a lot of discrete absorption lines in an infrared wavelength region. In the embodiment, by using infrared light in a wavelength range of 4.34 μm (corresponding to a wavenumber of 2300 cm⁻¹) or more to 4.39 μm (corresponding to a wavenumber of 2280 cm⁻¹) or less in a wide infrared wavelength region, a gas concentration of about 8% or less in breath (here, expired air) is measured.

In order to tune the wavelength of infrared light to an absorption line, a wavelength-tunable light-emitting element 10b is used. For example, in the case where the light-emitting element 10b is a QCL, an emission wavelength slightly varies depending on a current or a temperature. Due to this, by changing a driving current for the QCL with respect to a time, the wavelength can be tuned to an absorption line. Alternatively, also by changing the temperature of the QCL by a Peltier element or the like, it can be tuned to an absorption line.

Next, the gas cell 20 will be described in detail. The amount of exhaled breath by human beings is about 500 mL/breath. When the volume of the gas cell 20 is larger than 500 mL, it is affected by breath which is not exchanged in the lung (dead space air), and the detection performance is decreased. That is, it is preferred to set the volume of the gas cell 20 to 500 mL or less. Further, the shape of the gas cell 20 can be, for example, a circular columnar shape (the volume is about 4 mL) having a diameter of 16 mm and a cell length L of 20 mm, or the like.

The gas cell 20 can include an inlet 22 for breath BR or a reference gas (air or the like) having a valve 23 and an outlet 24 having a valve 25. When a vacuum pump (not shown) is connected to the outlet 24 and the pressure is reduced, a line width of an absorption line is decreased, and an overlap between adjacent absorption lines is reduced. Therefore, the absorption lines of CO₂ isotopes can be separated. The incident surface 20a and the emission surface 20b of the gas cell 20 can be window portions or the like having a high infrared light transmittance.

The gas measuring apparatus can further include a thermostat 90 in which the gas cell 20 is internally stored. The thermostat 90 maintain the inside of the gas cell 20 at a constant temperature by, for example, providing a heater or the like on a side surface of the gas cell 20, and further surrounding or the like the circumference thereof with a thermal insulation material. An absorption coefficient α of a gas varies depending on the temperature of the gas, and therefore, by maintaining the gas cell 20 at a constant temperature, the gas concentration measurement precision can be increased. Further, as shown in the drawing, small diameter pipes 26a, 26b, 26c, 26d, and 26e are arranged by being diverted from the inlet 22 to a measured portion 28, the temperature of the gas when incident on the measured portion 28 can be brought close to the temperature in the gas cell 20.

In the case where a reference gas such as air is introduced into the gas cell 20 of the gas measuring apparatus, a first transmittance can be calculated assuming that an intensity of light transmitted through the reference gas introduced into the gas cell 20 at a first wavelength is equal to an incident light intensity. Further, a second transmittance can be calculated assuming that an intensity of light transmitted through the reference gas at a second wavelength is equal to an incident light intensity. A step of measuring a target gas and a step of measuring the reference gas are alternately performed a plurality of times. Further, it is possible to increase the concentration measurement precision by averaging measured signal values.

The detection portion 40 can include a light receiving portion 40a or a data processing portion 40b. The light receiving portion 40a can be a photodiode or a cooling-type detector (MCT: composed of HgCdTe) or the like.

The gas measuring apparatus can further include an incident portion 50 which is provided contiguous to the incident surface 20a and collimates infrared light emitted from the light-emitting element 10b, and an emission portion 60 which is provided contiguous to the emission surface 20b and converges the parallel light toward the detection portion. A lens 50a on a gas cell 20 side is formed into a planar shape, and a lens 60a on a gas cell 20 side is formed into a planar shape, whereby an optical path length of parallel light passing through a target gas is made constant, and the measurement precision can be further increased. In this manner, the optical path length passing through air can be set to, for example, 7 mm or less, and an effect of an interfering gas in air or the like can be decreased.

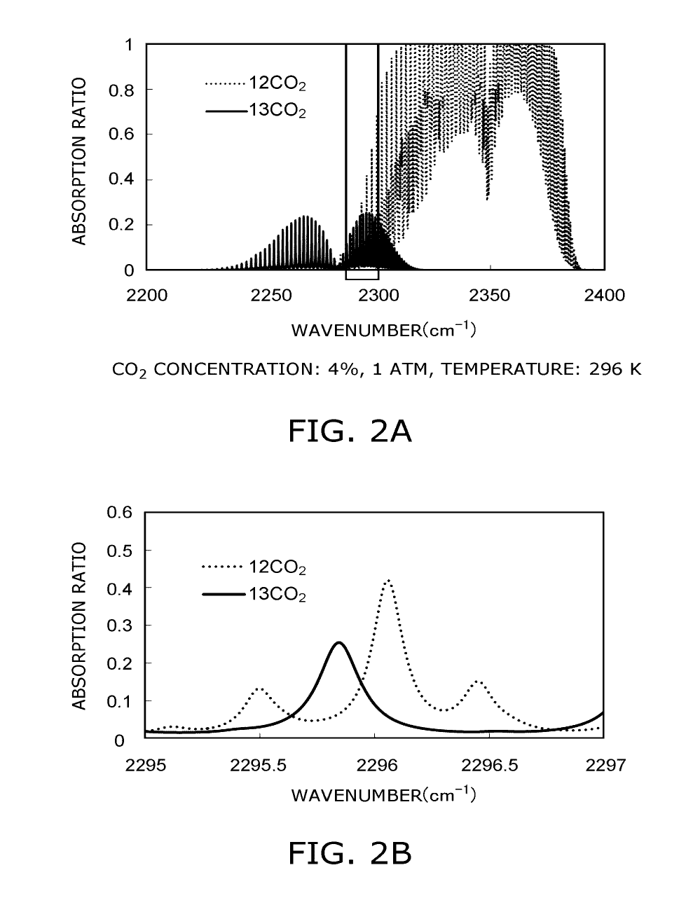

FIG. 2A is a graph showing an absorption ratio at a wavenumber of 2200 to 2400 cm⁻¹, and FIG. 2B is a graph showing an absorption ratio at a wavenumber of 2295 to 2297 cm⁻¹.

The vertical axis represents an absorption ratio, and the horizontal axis represents a wavenumber (cm⁻¹). Further, the measurement conditions are as follows: CO₂ concentration: 4%, pressure: 1 atm, temperature: 296 K.

First, the Lambert-Beer law is represented by the formula (1). This law has high precision for a thin gas, however, as a gas concentration is higher, absorption is saturated, and therefore, correction is needed. Incidentally, the absorption coefficient α is determined by an absorption line intensity, a pressure, and a temperature.

A=−In(I/I₀)=−InT=αL=εcL (1)

where A is absorbance, I₀ is incidence light intensity, I is transmitted light intensity, T is transmittance, α is absorption coefficient, and L is optical path length.

Incidentally, in FIGS. 2A and 2B, the absorption ratio is represented by the following formula.

Absorption ratio=1−I/I₀=1−T

As shown in FIG. 2A, the absorption ratio extends in a wide range, and ¹³CO₂ and ¹²CO₂ overlap with each other, and the spectral shapes are different. For example, when an infrared lamp (which may be combined with a filter) is used as a light source, it is necessary to perform data processing by measuring a lot of absorption lines constituting an extended spectrum. Further, when the wavenumber is from 2300 to 2360 cm⁻¹, the absorption ratio comes closer to 1, and absorption becomes close to saturation. Due to this, the deviation from the Lambert-Beer law becomes large, and the necessity to perform correction using a calibration curve table or the like occurs. As a result, data processing becomes complicated, and the measurement error is increased.

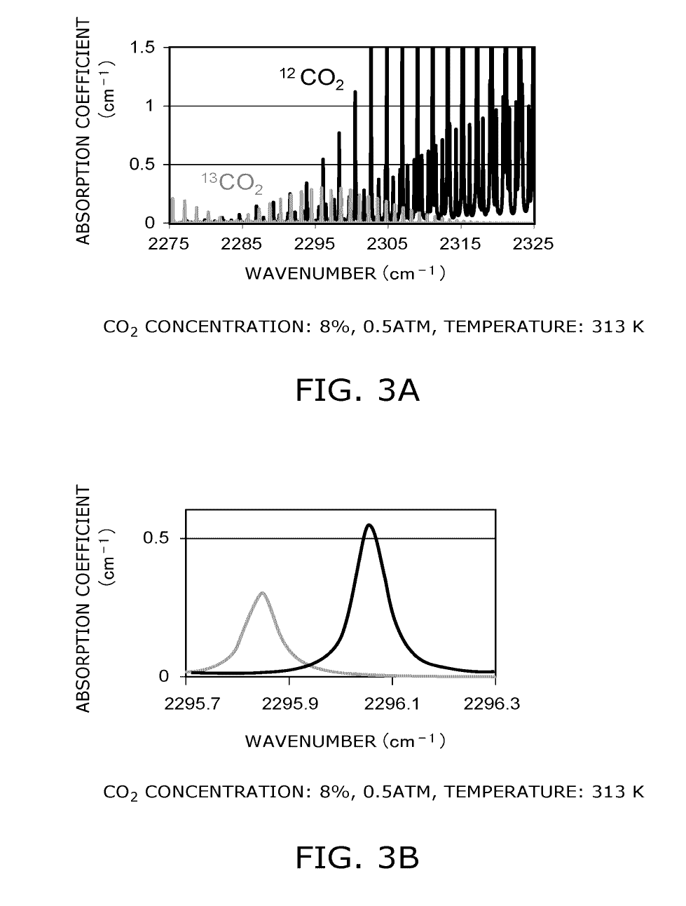

FIG. 3A is a graph showing the absorption coefficients of ¹³CO₂ and ¹²CO₂ at a wavenumber of 2275 to 2325 cm⁻¹, and FIG. 3B is a graph showing the absorption coefficients at a wavenumber of 2295.7 to 2296.3 cm⁻¹.

Incidentally, the CO₂ concentration is set to 8%, the pressure is set to 0.5 atm, and the temperature is set to 313 K.

The concentration of CO₂ contained in human breath is from 0.5 to 8% or so. On the other hand, in the embodiment, by narrowing the wavelength range to 2280 to 2300 cm⁻¹, and also maintaining the absorption ratio at less than 1, the absorption ratios of ¹³CO₂ and ¹²CO₂ are made substantially the same at a natural isotope ratio. Therefore, the measurement error can be reduced. Further, FIG. 3B shows a wavenumber range including one absorption line of each isotope. The absorption coefficient α is 0.55 cm⁻¹ or less.

Next, a method for detecting Helicobacter pylori using isotopes will be described. For example, a person takes a reagent containing ¹³C-urea as a labeled compound. When Helicobacter pylori is present in the stomach, the reagent and Helicobacter pylori react with each other, and ¹³CO₂ is exhaled as expired air. On the other hand, when Helicobacter pylori is not present, ¹³CO₂ is not exhaled. Due to this, by measuring an isotope ratio of ¹³CO₂ to ¹²CO₂, the degree of infection with Helicobacter pylori can be known, and thus, gastric diagnosis can be made with high precision. Incidentally, a test target is not limited to Helicobacter pylori. By measuring the concentration of CO₂ including isotopes, gastric clearance can be diagnosed in a wide range.

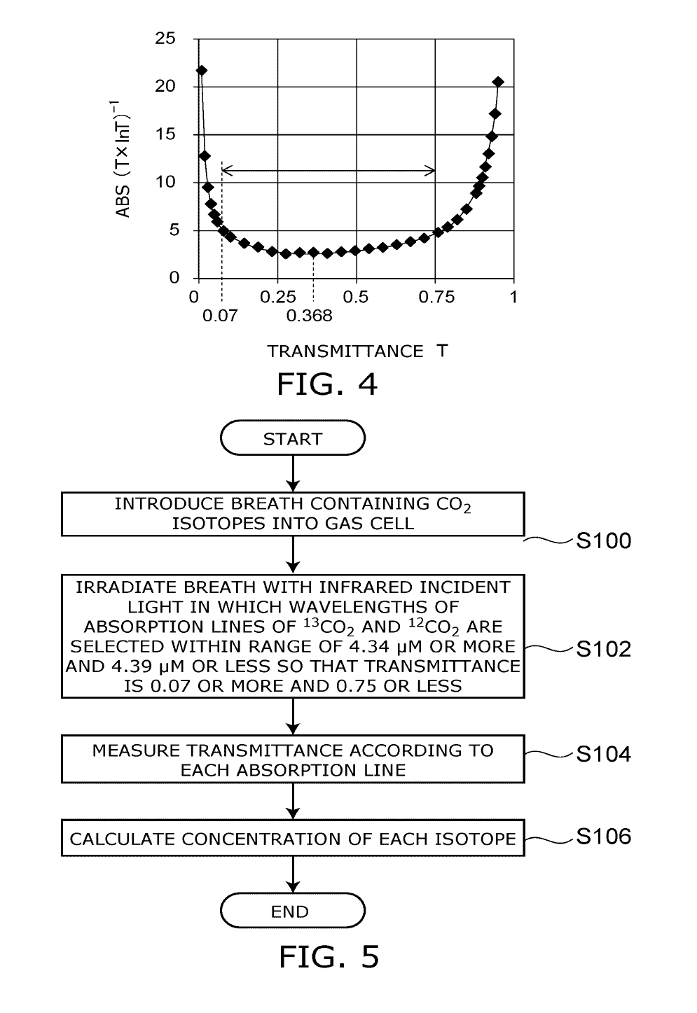

FIG. 4 is a graph showing dependence of the relative error of a gas concentration on a transmittance.

First, the absorption coefficient α can be represented by the formula (2).

α=εc (2)

where ε is molar extinction coefficient.

When using the formula (1) and the formula (2), a molar concentration c is represented by the formula (3).

c=−(1/εL)×InT (3)

where c is molar concentration.

A changing ratio of the molar concentration c to the transmittance T can be represented by the formula (4).

dc/dT=−(1/εL)×1/T (4)

dc/c is represented by the formula (5). The relative error can be defined as an absolute value (ABS) of dc/c and is determined by a function (T×InT)⁻¹.

dc/c=(1/T×InT)×dT (5)

As shown in FIG. 4, there exists a minimum value in the relative error. The relative error is minimized when d(T×InT)⁻¹/dT=0. The condition therefor is represented by the formula (6).

InT+1=0 (6)

According to the formula (6), the transmittance T when the relative error is minimized is represented by the formula (7).

Top=1/e (7)

where Top is transmittance at which relative error is minimized and e is Napier's constant.

That is, when Top=1/e, the absorbance A=−InT=1.

FIG. 5 is a flow diagram of the gas measuring method according to the embodiment.

The gas measuring method includes the steps of introducing breath containing ¹³CO₂ and ¹²CO₂ into the gas cell 20 having a cell length L of 2.5 cm or more and 20 cm or less (S100), irradiating the breath BR with infrared incident light GI in which the wavelengths of an absorption line of ¹³CO₂ at the first wavelength and an absorption line of ¹²CO₂ at the second wavelength are selected within a wavelength range of 4.34 μm or more and 4.39 μm or less so that the transmittance T is 0.07 or more and 0.75 or less (S102), measuring a first transmittance of light transmitted through the gas cell 20 at the first wavelength and a second transmittance of light transmitted through the gas cell 20 at the second wavelength (S104), and calculating the concentrations of ¹³CO₂ and ¹²CO₂ (S106).

Incidentally, the gas measuring method can further include the steps of introducing a reference gas into the gas cell 20, calculating a first transmittance assuming that an intensity of light transmitted through the reference gas at a first wavelength is equal to an incident light intensity, and calculating a second transmittance assuming that an intensity of light transmitted through the reference gas at a second wavelength is equal to an incident light intensity.

There is an individual difference in the concentration of CO₂ in human breath, and the concentration of CO₂ is from about 0.5 to 8%. Further, a median value thereof is about 4%. According to the measurement made by the inventor, when the concentration of CO₂ is 4%, the absorption coefficient α at a wavenumber of 2300 to 2380 cm⁻¹ was from 0.05 to 0.4 cm⁻¹ (provided that the pressure was 0.5 atm, and the temperature was 313 K). At this time, an optimal gas cell length Lop is represented by the formula (8).

Lop=−InTop/α=1/α (8)

In (Table 1), the optimal gas cell length Lop with respect to the absorption coefficient α is shown.

TABLE 1

| α (cm⁻¹) | Lop (cm) |
|---|---|
| 0.05 | 20.0 |
| 0.1 | 10.0 |
| 0.2 | 5.0 |
| 0.3 | 3.3 |
| 0.4 | 2.5 |

That is, when the absorption coefficient α is set to 0.05 to 0.4 cm⁻¹, the optimal gas cell length Lop may be set to 2.5 cm or more and 20 cm or less.

When the measurement error of the transmittance T is constant, the transmittance T is 1±0.5%, the width of the absorbance A is from 4.2 to 5.3, and the error is increased to 23.85%. Further, when the transmittance T is 99±5%, the width of the absorbance A is from 0.005 to 0.015, and the error reaches 100% or more. On the other hand, when the transmittance T measured using the gas cell 20 in which the cell length is selected from the range of 2.5 cm to 20 cm (at 4.34 to 4.39 μm) is between 0.07 and 0.75, the measurement error is decreased to 5% or less. Due to this, the precision of measurement of a gas concentration c can be kept high.

Next, the QCL to be used as the light source will be described.

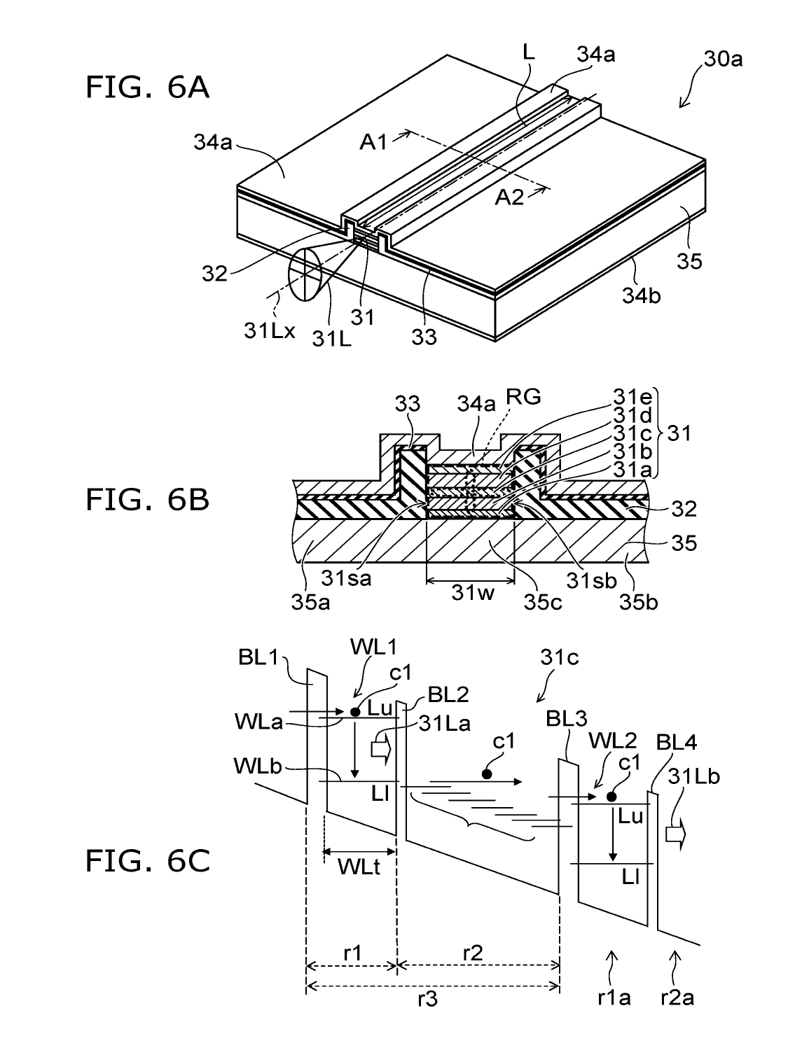

FIGS. 6A to 6C are schematic views of the QCL.

FIG. 6A is a schematic perspective view of the QCL. FIG. 6B is a schematic cross-sectional view taken along the line A1-A2 of FIG. 6A. FIG. 6C is a schematic view illustrating an operation of the QCL.

In this example, as the light source 10, a semiconductor light-emitting element 30aL which is the QCL is used.

As shown in FIG. 6A, the semiconductor light-emitting element 30aL includes a substrate 35, a stacked body 31, a first electrode 34a, a second electrode 34b, a dielectric layer 32 (first dielectric layer), and an insulating layer 33 (second dielectric layer).

Between the first electrode 34a and the second electrode 34b, the substrate 35 is provided. The substrate 35 includes a first portion 35a, a second portion 35b, and a third portion 35c. These portions are disposed in the same one plane. This plane crosses (for example, is parallel) in a direction from the first electrode 34a to the second electrode 34b. Between the first portion 35a and the second portion 35b, the third portion 35c is disposed.

Between the third portion 35c and the first electrode 34a, the stacked body 31 is provided. Between the first portion 35a and the first electrode 34a, and between the second portion 35b and the first electrode 34a, the dielectric layer 32 is provided. Between the dielectric layer 32 and the first electrode 34a, the insulating layer 33 is provided.

The stacked body 31 has a stripe shape. The stacked body 31 functions as a ridge waveguide RG. Two end faces of the ridge waveguide RG become mirror faces. Light 31L emitted in the stacked body 31 is emitted from the end face (light emission face). The light 31L is infrared laser light. An optical axis 31Lx of the light 31L is along an extending direction of the ridge waveguide RG.

As shown in FIG. 6B, the stacked body 31 includes, for example, a first clad layer 31a, a first guide layer 31b, an active layer 31c, a second guide layer 31d, and a second clad layer 31e. These layers are arranged in this order along a direction from the substrate 35 to the first electrode 34a. Each of the refractive index of the first clad layer 31a and the refractive index of the second clad layer 31e is lower than each of the refractive index of the first guide layer 31b, the refractive index of the active layer 31c, and the refractive index of the second guide layer 31d. The light 31L generated in the active layer 31c is confined in the stacked body 31. The first guide layer 31b and the first clad layer 31a are sometimes combined and called “clad layer”. The second guide layer 31d and the second clad layer 31e are sometimes combined and called “clad layer”.

The stacked body 31 has a first side surface 31sa and a second side surface 31sb perpendicular to the optical axis 31Lx. A distance 31w (width) between the first side surface 31sa and the second side surface 31sb is, for example, 5 μm or more and 20 μm or less. According to this, for example, the control in a horizontal transverse direction mode is facilitated, and the improvement of output is facilitated. When the distance 31w is excessively long, a higher-order mode is likely to occur in a horizontal transverse direction mode, and it is difficult to increase the output.

The refractive index of the dielectric layer 32 is lower than the refractive index of the active layer 31c. According to this, the ridge waveguide RG is formed along the optical axis 31Lx by the dielectric layer 32.

As shown in FIG. 6C, the active layer 31c has, for example, a cascade structure. In the cascade structure, for example, a first region r1 and a second region r2 are alternately stacked. A unit structure r3 includes the first region r1 and the second region r2. A plurality of unit structures r3 is provided.

For example, in the first region r1, a first barrier layer BL1 and a first quantum well layer WL1 are provided. In the second region r2, a second barrier layer BL2 is provided. For example, in another first region r1a, a third barrier layer BL3 and a second quantum well layer WL2 are provided. In another second region r2a, a fourth barrier layer BL4 is provided.

In the first region r1, optical transition between subbands in the first quantum well layer WL1 occurs. Due to this, for example, light 31La having a wavelength of, for example, 3 μm or more and 18 μm or less is emitted.

In the second region r2, energy of a carrier c1 (for example, an electron) injected from the first region r1 can be relaxed.

In the quantum well layer (for example, the first quantum well layer WL1), a well width WLt is, for example, 5 nm or less. When the well width WLt is narrow in this manner, an energy level is discrete, and for example, a first subband WLa (a high level Lu) and a second subband WLb (a low level LI), or the like occur. The carrier c1 injected from the first barrier layer BL1 is effectively confined in the first quantum well layer WL1.

When the transition of the carrier c1 from a high level Lu to a low level LI occurs, the light 31La corresponding to the difference in energy (the difference between the high level Lu and the low level LI) is emitted. That is, optical transition occurs.

Similarly, in the second quantum well layer WL2 in another first region r1a, light 31Lb is emitted.

In the embodiment, the quantum well layer may include a plurality of wells whose wave functions overlap each other. The high levels Lu of the respective plurality of quantum well layers may be the same as each other. The low levels LI of the respective plurality of quantum well layers may be the same as each other.

For example, optical transition between subbands occurs in either of a conduction band and a valence band. For example, recombination of a hole and an electron by p-n junction is not needed. For example, optical transition occurs by the carrier c1 of either of a hole and an electron, and light is emitted.

In the active layer 31c, for example, by a voltage applied between the first electrode 34a and the second electrode 34b, the carrier c1 (for example, an electron) is injected into the quantum well layer (for example, the first quantum well layer WL1) through the barrier layer (for example, the first barrier layer BL1). According to this, optical transition between subbands occurs.

The second region r2 has, for example, a plurality of subbands. The subband is, for example, a miniband. The difference in energy in the subbands is small. It is preferred that the subbands are close to continuous energy bands. As a result, the energy of the carrier c1 (electron) is relaxed.

In the second region r2, for example, light (for example, infrared light having a wavelength of 3 μm or more and 18 μm or less) is substantially not emitted. The carrier c1 (electron) at a low level LI in the first region r1 passes through the second barrier layer BL2 and is injected into the second region r2 and relaxed. The carrier c1 is injected into another first region r1a connected in cascade. In this first region r1a, optical transition occurs.

In the cascade structure, optical transition occurs in each of the plurality of unit structures r3. According to this, it becomes easy to obtain a high light output in the entire active layer 31c.

In this manner, the light source 10 includes the semiconductor light-emitting element 30aL. The semiconductor light-emitting element 30aL emits measurement light 30L by energy relaxation of electrons in the subbands in the plurality of quantum wells (for example, the first quantum well layer WL1 and the second quantum well layer WL2, etc.).

In the quantum well layers (for example, the first quantum well layer WL1 and the second quantum well layer WL2, etc.), for example, InGaAs is used. For example, in the barrier layers (for example, the first to fourth barrier layers BL1 to BL4, etc.), for example, InAlAs is used. At this time, for example, when InP is used as the substrate 35, favorable lattice matching is obtained in the quantum well layers and the barrier layers.

The first clad layer 31a and the second clad layer 31e contain, for example, Si as an n-type impurity. The concentration of the impurity in these layers is, for example, 1×10¹⁸ cm⁻³ or more and 1×10²⁰ cm⁻³ or less (for example, about 6×10¹⁸ cm⁻³). The thickness of each of these layers is, for example, 0.5 μm or more and 2 μm or less (for example, about 1 μm).

The first guide layer 31b and the second guide layer 31d contain, for example, Si as an n-type impurity. The concentration of the impurity in these layers is, for example, 1×10¹⁶ cm⁻³ or more and 1×10¹⁷ cm⁻³ or less (for example, about 4×10¹⁶ cm⁻³). The thickness of each of these layers is, for example, 2 μm or more and 5 μm or less (for example, about 3.5 μm).

The distance 31w (the width of the stacked body 31, that is, the width of the active layer 31c) is, for example, 5 μm or more and 20 μm or less (for example, about 14 μm).

The length of the ridge waveguide RG is, for example, 1 mm or more and 5 mm or less (for example, about 3 mm). The semiconductor light-emitting element 30aL operates at an operation voltage of, for example, 10 V or less. The current consumption is lower than a carbon dioxide gas laser apparatus or the like. According to this, an operation with low power consumption can be achieved.

According to the gas measuring apparatus and the gas measuring method of the embodiment, a gas measuring apparatus and a gas measuring method in which a measurement error is reduced can be provided.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A gas measuring apparatus, comprising:
a light source which emits infrared light;
a gas cell, into which gas containing ¹³CO₂ and ¹²CO₂ is introduced, and which includes an incident surface on which the infrared light is incident and an emission surface through which the infrared light is transmitted, and has a cell length of 2.5 cm or more and 20 cm or less; and
a detection portion which measures a first transmittance of transmitted light from the emission surface at a first wavelength in wavelength range in an absorption line of the ¹³CO₂ and a second transmittance of transmitted light from the emission surface at a second wavelength in the wavelength range in an absorption line of the ¹²CO₂, and is capable of calculating a concentration of each of the ¹³CO₂ and the ¹²CO₂.

2. The apparatus according to claim 1, further comprising:
an incident portion which is provided contiguous to the incident surface and collimates the infrared light; and
an emission portion which is provided contiguous to the emission surface and converges the parallel light.

3. The apparatus according to claim 2, wherein an optical axis of the infrared light is orthogonal to both of the incident surface and the emission surface.

4. The apparatus according to claim 1, wherein the infrared light is tunable in a wavelength range of 4.34 μm or more to 4.39 μm or less.

5. The apparatus according to claim 4, wherein the infrared light is emitted from a quantum cascade laser.

6. The apparatus according to claim 1, wherein the detection portion calculates the first transmittance assuming that an intensity of light transmitted through a reference gas introduced into the gas cell at the first wavelength is equal to an incident light intensity, and calculates the second transmittance assuming that an intensity of light transmitted through the reference gas at the second wavelength is equal to an incident light intensity.

7. The apparatus according to claim 6, further comprising:
an incident portion which is provided contiguous to the incident surface and collimates the infrared light; and
an emission portion which is provided contiguous to the emission surface and converges the parallel light.

8. The apparatus according to claim 1, wherein a volume of the gas cell is 500 mL or less.

9. The apparatus according to claim 1, wherein an inside of the gas cell can be decompressed.

10. The apparatus according to claim 1, further comprising a thermostat in which the gas cell is internally stored.

11. A gas measuring method, comprising:
Introducing gas containing ¹³CO₂ and ¹²CO₂ into a gas cell having a cell length of 2.5 cm or more to 20 cm or less;
irradiating the gas with infrared incident light;
measuring a first transmittance of light transmitted through the gas cell at the first wavelength and a second transmittance of light transmitted through the gas cell at the second wavelength; and
calculating concentrations of the ¹³CO₂ and the ¹²CO₂.

12. The method according to claim 11, wherein the gas is introduced into the gas cell which is decompressed.

13. The method according to claim 11, wherein in the infrared incident light, wavelengths of an absorption line of the ¹³CO₂ at the first wavelength and an absorption line of the ¹²CO₂ at the second wavelength are selected within a wavelength range of 4.34 μm or more to 4.39 μm or less so that a transmittance is 0.07 or more and 0.75 or less.

14. A gas cell, which comprises:
an incident surface of infrared light;
an emission surface of the infrared light;
an inlet for gas and a reference gas, having a first valve; and
an outlet for the gas and the reference gas, having a second valve, and in which
a cell length is 2.5 cm or more and 20 cm or less, and a volume when the first valve and the second valve are brought to a closed state is 500 mL or less.
